(19) United States
(12) Patent Application Publication
(10) Pub. No.: US 2005/0230181 A1
(43) Pub. Date: Oct. 20, 2005

(54) Length adjustable earplug cord

(76) Inventor: Edwin Woo, Chula Vista, CA (US)

Correspondence Address: LEON D. ROSEN;FREILICH, HORNBAKER & ROSEN, Suite 1220, 10960 Wilshire Boulevard, Los Angeles, CA, 90024, US

(21) Appl. No.: 11/100,942

(22) Filed: Apr. 7, 2005

Related U.S. Application Data
(60) Provisional application No. 60/567,122, filed on Apr. 15, 2004.

(51) Int. Cl. H04R 25/02; A61B 7/02

(52) U.S. Cl. 181/135; 181/130

(57) ABSTRACT

A cord-earplug set (10) which includes a cord (16) that connects together a pair of earplugs (12, 14), the cord having a length that can be readily increased by a worker who pulls apart the opposite ends of the cord. The cord has an initial length much less than the usual two feet, such as 11 to 14 inches, and is constructed so the cord can be stretched by a worker pulling apart the cord ends with a force on the order of magnitude of 15 pounds. The cord is preferably formed of an elastomeric polymer of uniform diameter along its length so when the cord ends are pulled apart (by pulling apart the earplugs) to stretch the cord, the cord sections lengthen evenly to leave a cord of approximately constant diameter along its length.

### CROSS-REFERENCE

Applicant claims priority from U.S. patent application SN 60/567,122 filed Apr. 15, 2004.

### BACKGROUND OF THE INVENTION

Earplugs are commonly worn by workers who are subjected to loud noise, to protect their hearing. In contamination-sensitive industries such as in the preparation of food and medicine, and where delicate machinery could be damaged by an earplug, it is important to minimize the possibility that an earplug will fall into the material or machinery. This is commonly done by tying together a pair of earplugs with a cord. In most instances where an earplug may drop, the cord will stop its fall as where the other earplug of the pair is held in an ear or the cord is separately held.

Presently the length of the cord is about two to two and one-half feet, which is long enough to allow it to be worn by almost all workers. However, for some workers, a cord length of two to two one-half feet is too long. A cord of more than one and one-half feet length may dangle behind a person's back and may present the danger that the cord will catch on a protruding piece of equipment. In fact, a person who repairs machinery in a noisy environment may desire a cord length such as 11¼ inches, to wear the cord against the back of his/her neck and without appreciable slack. On the other hand, a person with a “puffy” hair style and who wears a bulging hair covering over and behind the head, may want a cord of two to two and one-half feet length to easily move the cord around the hair or covering. A cord that allowed a user to easily achieve the desired cord length would be of value.

### SUMMARY OF THE INVENTION

In accordance with one embodiment of the invention, a corded earplug set containing a cord and two earplugs is provided wherein the cord is initially shorter than prior cords of two feet length, such as no more than 11 to 16 inches length. However, the cord is permanently, or plastically elongatable by more than an inch and preferably by at least 10%, by a tension force on the order of magnitude of 15 pounds (1.5 to 150 pounds), that most workers can apply. This is preferably done by forming the cord of elastomeric material of uniform characteristics along the entire length of the cord between the earplugs so cord thinning is uniform as it stretches.

The opposite ends of the cord each extends into a passage in the rear portion of a corresponding earplug. The cord is preferably stretched by the wearer grasping the rear ends of the two earplugs, and then pulling the earplugs apart. By grasping the earplugs the person avoids harm to his/her hands, prevents the cord from pulling out of the earplugs, and assures that the maximum and constant length of cord is being stretched.

The novel features of the invention are set forth with particularity in the appended claims. The invention will be best understood from the following description when read in conjunction with the accompanying drawings.

### BRIEF DESCRIPTION OF THE DRAWINGS

FIG. 1 is a partially sectional side elevation view of a corded earplug combination of one embodiment of the invention.

FIG. 2 is a rear isometric view of a person wearing the corded earplug combination of FIG. 1 with the cord being of a short length, and with other short cord length positions shown in phantom lines.

FIG. 3 is a rear isometric view of another person wearing the corded earplug of FIG. 1 wherein the cord has been lengthened.

FIG. 4 is a partially sectional side elevation view of a corded earplug combination of another embodiment of the invention.

### DESCRIPTION OF THE PREFERRED EMBODIMENTS

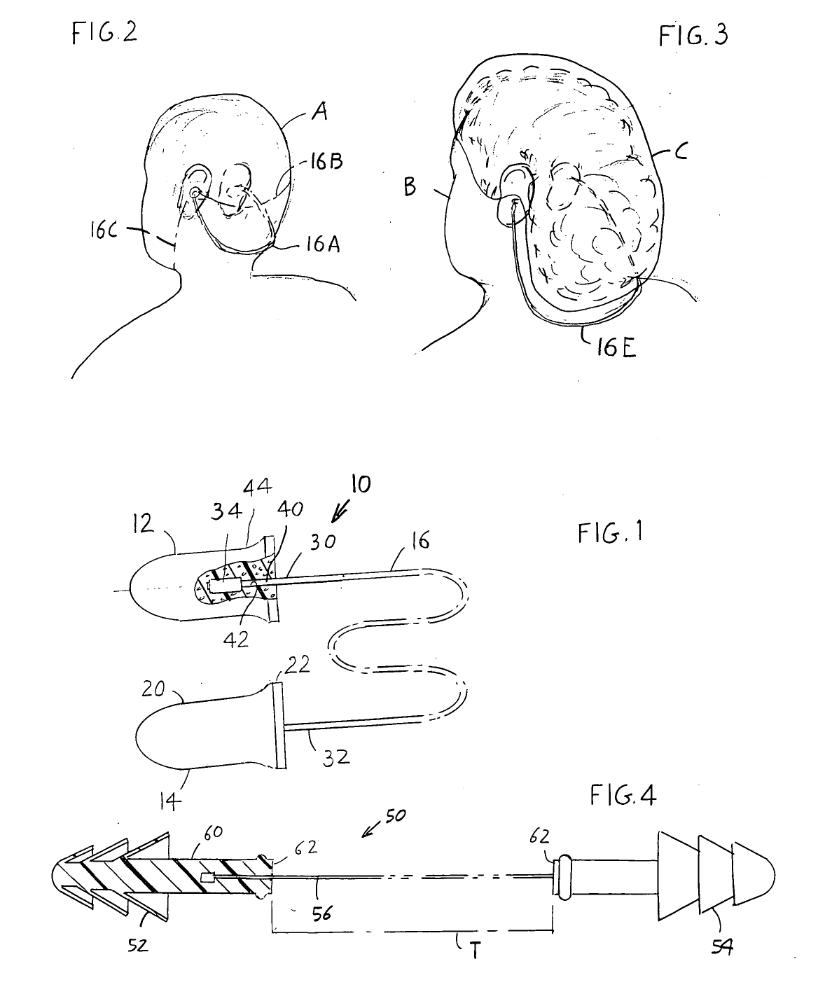

FIG. 1 illustrates a corded earplug combination 10 which includes a pair of earplugs 12, 14 and a cord 16 that joins the earplugs. Each earplug has a front portion 20 that is pressed into a wearer's ear canal and a rear portion 22 that typically has a major part that remains outside the ear canal. The particular earplugs 14, 16 illustrated are each formed of slow recovery elastomeric foam material and may be rolled to a small diameter before it is inserted into the ear canal where it expands and blocks noise.

The cord 16 has opposite ends 30, 32 that are each fixed to one of the earplugs. In FIG. 1, a fitting 34 has been fixed around each end of the cord. The fitting 34 and a length 40 of the cord beyond the fitting lie in a passage 42 of the earplug rear portion and are fixed in place therein by the fitting lying in a large interference fit in the passage. Adhesive can be used instead of or in addition to the fitting to securely hold the cord in the passage.

Prior art cords of a corded earplug combination typically have a length between the earplugs of about two feet (24 inches or 61 centimeters) to two and one-half feet (30 inches or 76 cm). This assures that the cord will be long enough for all applications. Applicant finds that there are occasions when a worker wishes to have a much shorter cord. FIG. 2 illustrates a person A who wears the corded earplug combination with a cord at 16A that extends almost tightly around the back of the person's head. The shortest cord would be one that extends tightly around the back of the head as at 16B, or where it extends around the neck at 16C or even around the top of the head. Such short cords at 16A to 16C, which have a length of about 11 to 16 inches, can be useful to assure that the cord will not catch on protruding parts in the work environment, and can be especially useful for a machinery repairman who moves close to protruding parts.

FIG. 3 illustrates a worker B with longer hair that has been enclosed in a head covering C to help maintain a very clean environment. Such a person may want to wear a corded earplug combination with a long cord 16E such as one that is 24 inches long, which does not press against the head covering.

In accordance with the present invention, the cord 16 is initially supplied so it has a short length such as no more than 14 to 16 inches, such as about 11 inches. However, the cord is of a material and construction that allows the cord to be readily plastically elongated (it retains its elongation when the stretching force is removed) by at least 10%, preferably at least 20% and more preferably at least 100% of its original length by the person who will wear the combination. Prior art cords were formed of flexible polymers that could not be elongated at least 10% without breaking, that required larger forces than a typical worker could apply by hand to elongate it by even 8%, and which usually broke before elongation of 8%).

Applicant constructs the cord 16 of the present invention of a selected material and of a cord cross sectional area that allows the cord to be readily elongated by force applied the hands of a worker. Applicant prefers to construct the cord of a thermoplastic elastomeric polymer. Flexible polymers commonly have a Young's modulus of elasticity (in tension) of less than 1000×10³ and usually less than 500×10³. Elastomers have a Young's modulus (in tension) of no more than 50×10³. By contrast, iron has a Young's modulus of about 30,000×10³.

The cord 16 is formed of a flexible material that resiliently elongates when stretched by up to its elastic limit, and that thereafter plastically elongates. When the stretching forces are no longer applied, the cord length decreases slightly, but retains much of its elongation. In one example, the cord has an initial length of 11¼ inches (29 cm). A person “tries on” the combination ( but initially without the earplugs inserted into the ears) and may find it to be slightly too short. The person removes the combination and pulls apart opposite ends of the cord to stretch it to perhaps 14 inches, and it recovers to 12 inches. The person tries on the combination and may repeat the process until the length is satisfactory for that person. A person can sense when the cord has been stretched beyond its elastic limit because resistance to further stretching does not increase. In most situations where workers regularly wear the combination of earplugs and a cord, the person often takes a new combination from a bin, such as every day or week, and becomes used to the amount of stretching to apply.

FIG. 1 shows that the end or end portion 40 of the cord that lies in the earplug, has a length of about one-half inch (1 cm) which is perhaps one-third to one-half the length of the earplug. Applicant provides instructions to workers to stretch the cord by grasping and squeezing the rear portion 44 of each earplug and pulling them apart. By grasping the earplugs instead of the cord ends, the worker avoids harm to his/her hands by the narrow cord. If the worker should grasp the front ends of the earplugs and pull them apart, the cord ends might pull out of the earplugs. A person can sense when the cord has stretched beyond its elastic limit. By having the worker grasp the earplug rear portions 44, applicant assures that a maximum of the cord length is stretched. Also, if a person stretches the cord several times, this results in all portions of the cord being stretched about the same amount to help avoid localized thinning. If a person pulls apart different parts of the cord at different times, then this contributes to uneven cord thinning.

The cord is formed of a flexible material that is a thermoplastic polymer which resiliently elongates when stretched by up to its elastic limit, which may be 20% of its initial length. However, the cord permanently deforms in a uniform manner when stretched by more than its elastic limit, up to at least 100% of its initial length. Such uniform stretching is obtained by assuring that the material to be extruded is very uniform in characteristics, as by very thorough mixing of all components prior to extrusion, by extruding a cord of uniform diameter, and by allowing all portions of the extruded cord to solidify in a uniform manner.

FIG. 4 illustrates another combination 50 of earplugs 52, 54 and cord 56, wherein the earplugs are each formed of a non-foam elastomer. The earplugs have long stems 60 and the cord ends lie only in the stems. The cord length T between earplug rear ends 62 is about 11 inches. The cord is of the same construction as for the combination of FIG. 1.

Thus, the invention provides a cord and earplug set or combination, wherein the cord can be individually adjusted in length, especially by the user. The cord is formed of a polymer that can be plastically (permanently) elongated by at least 20%, preferably at least 50%, and most preferably by at least 100% without breaking. The elongation is achieved by pulling apart opposite ends of the cord until plastic stretching is sensed. The material and thickness of the cord are chosen so that the cord can be stretched by an average worker, and the required force is on the order of magnitude of 15 pounds. Applicant prefers to form the cord of an elastomeric polymer.

Although particular embodiments of the invention have been described and illustrated herein, it is recognized that modifications and variations may readily occur to those skilled in the art, and consequently, it is intended that the claims be interpreted to cover such modifications and equivalents.

1. A cord-earplug set which includes a pair of earplugs that each has front and rear ends, and a flexible cord that extends between rear ends of said earplugs so the cord can extend behind a person in its extension between said earplug rear ends, wherein:
said cord is permanently stretchable by more than an inch by a tension force on the order of magnitude of fifteen pounds.

2. The cord-earplug set described in claim 1 wherein:
said cord has an initial length of no more than 16 inches.

3. The cord-earplug set described in claim 1 wherein:
said cord has an initial length of about 11 inches.

4. The cord-earplug set described in claim 1 wherein:
said cord is formed of elastomeric material and is of uniform material and diameter along its length between the rear ends of said earplugs.

5. A cord-earplug set which includes a pair of earplugs that each has front and rear ends, and a flexible cord that extends between rear ends of said earplugs so the cord can extend behind a person in its extension between said earplug rear ends, wherein:
said cord has an initial length of no more than about 16 inches, but is permanently stretchable by at least an inch by an average wearer pulling apart opposite end portion of the cord using only the wearer's hands.

6. The cord-earplug set described in claim 5 wherein:
said earplug is formed of elastomeric material.

7. A method for producing a cord-earplug set and adjusting it to an individual, where the cord-earplug set includes a pair of earplugs that each has front and rear end portions, and also includes a flexible cord that extends between said rear ends of said earplugs, comprising:
constructing said cord with an initial length and with a construction that makes the cord permanently stretchable by more than 25% of its initial length by a pull force on the order of magnitude of 15 pounds.
applying a stretching tension to said cord and stretching it so it is permanently stretched by at least 10% of its initial length.

8. The method described in claim 7 wherein said steps of constructing includes inserting ends of said cord into said earplug rear portions, and wherein:
said step of applying a stretching tension includes grasping and squeezing the rear end of each of said earplugs, to prevent cord pullout therefrom, while pulling apart said earplugs to stretch said cord.
